(19) United States
(12) Patent Application Publication
(10) Pub. No.: US 2023/0391745 A1
(43) Pub. Date: Dec. 7, 2023

(54) SMALL MOLECULE DEGRADERS OF THE BROMODOMAIN AND PHD FINGER TRANSCRIPTION FACTOR

(71) Applicant: Regents of the University of Minnesota, Minneapolis, MN (US)

(72) Inventors: William Pomerantz et al., Minneapolis, MN (US); Huda Zahid, Minneapolis, MN (US); Jennifer Kimbrough, Minneapolis, MN (US)

(21) Appl. No.: 18/322,445

(22) Filed: May 23, 2023

Related U.S. Application Data
(60) Provisional application No. 63/344,929, filed on May 23, 2022.

(51) Int. Cl. C07D 401/12

(52) U.S. Cl. CPC C07D 401/12

(57) ABSTRACT

The disclosure relates to compounds of the formula A-L¹-B (Formula (I)) or a pharmaceutically acceptable salt, polymorph, prodrug, solvate or clathrate thereof, wherein A is a ligand for the bromodomains of at least one of BPTF, CECR2, BRD9, and PCAF/GCN5; B is an E3 ligase ligand; and L¹ is a linker.

### CROSS-REFERENCE TO RELATED APPLICATIONS

This application claims the benefit of U.S. Provisional Appl. Ser. No. 63/344,929, filed May 23, 2022, which is incorporated by reference as if fully set forth herein.

### STATEMENT OF U.S. GOVERNMENT SUPPORT

This invention was made with government support under grant GM140837-01 awarded by the National Institutes of Health. The government has certain rights in the invention.

### BACKGROUND

Epigenetic regulation occurs through mechanisms that modify gene expression without changing the genomic sequence. One such process is chromatin remodeling which involves alterations in the chromatin structure through changes in the nucleosome position or histone modification, eviction, or exchange. Remodeling can occur via both ATP-dependent and -independent mechanisms. The ATP-dependent processes are catalyzed by multidomain chromatin remodeling complexes classified into four families: SWI/SNF, ISWI, CHD and INO80. Dysregulation of these chromatin remodelers is often associated with oncogenic phenotypes. Several of these chromatin remodeling complexes also contain bromodomain-containing proteins. The mammalian SWI/SNF complexes BAF and PBAF contain the class IV bromodomains BRD9 and BRD7 subunits respectively. In addition, the GCN5 bromodomain stabilizes the SWI/SNF complex on chromatin. In the less-studied ISWI family, NURF recognizes chromatin through its largest subunit BPTF, and CERF contains the CECR2 bromodomain-containing protein. BPTF, CECR2 and GCN5 are members of the class I bromodomain family (FIG. 1B) and their role in nucleosome remodeling makes them important targets for anti-cancer therapeutic strategies.

Although not as well-characterized as BET inhibitors, several small-molecule inhibitors for class I and class IV bromodomains have been recently developed. However, for many non-BET bromodomains, it remains unclear whether bromodomain inhibition alone will be effective to induce a significant phenotypic effect. In the case of BPTF it has been shown that bromodomain inhibitors when used as single agents may be insufficient for anti-cancer therapeutic applications. It was previously reported that BPTF inhibitors sensitize 4T1 breast cancer cells to the chemotherapeutic drug doxorubicin, therefore combination treatment strategies may be useful options in cases where bromodomain inhibition on its own proves to be ineffective.

An alternative pharmacological modality is targeted protein degradation which has progressed rapidly for BET bromodomains. In contrast, only four non-BET bromodomain targeting degraders have been reported (FIG. 1A). These degraders have in some cases proven more effective than monovalent inhibitors. For example, the BRD9 inhibitor I-BRD9 demonstrated only modest effects in synovial sarcoma cells but a degrader generated from the same scaffold (dBRD9) led to a greater therapeutic response. Similarly, inhibition of the highly homologous PCAF/GCN5 bromodomains by the small-molecule probe GSK4027 was insufficient to recapitulate the effects of genetic knockouts in macrophages, motivating the development of the degrader GSK699. These studies highlight the significance of using protein degradation to target class I and class IV bromodomain-containing proteins.

### SUMMARY

Heterobifunctional molecules also provide a new way to establish selectivity across different bromodomains. Gadd et al. showed that the BRD4 degrader MZ1 can induce protein-protein interactions with the E3 ligase, leading to more stable and cooperative ternary complexes for BRD4 over other BET bromodomains. While designing selective small-molecule inhibitors for class I bromodomains remains challenging, forming distinct ternary complexes is a potential alternative for targeting specific members of the family. Targeted protein degradation may also be a useful therapeutic tool given that it allows the full protein to be removed through sub-stoichiometric treatment of degrader compounds in an event-driven process. For BPTF, genetic knockdown studies have shown strong downstream phenotypic effects. Richart et al. demonstrated that BPTF knockdown resulted in decreased c-Myc recruitment to DNA. BPTF knockdown also decreased high-grade glioma growth in adult and pediatric models. Another class I bromodomain, CECR2, was recently shown to drive breast cancer metastasis by activating the NF-κB pathway, making it a possible target for treating metastatic breast cancer. Given the therapeutic utility demonstrated by genetic knockdown studies, it is anticipated that protein degradation approaches would be valuable for modulating the activity of these proteins and the larger nucleosome remodeling complexes they form.

Described herein is the design and evaluation of first-generation degraders for class I bromodomains and BRD7/9 in class IV using two different scaffolds (FIG. 1C). A pyridazinone-based scaffold previously established for PCAF/GCN5 and BPTF, and a pyrimidine-based scaffold derived from TP-238, as a dual BPTF/CECR2 chemical probe were used. Exit vectors were established for linker attachment and ternary complex formation was explored through both in vitro assays and in-cell Nano-BRET. Focusing efforts on BPTF, in-cell NanoBRET assay was used to demonstrate degradation of a designed Nanoluciferase-bromodomain construct through both scaffolds and subsequently a full length Nanoluciferase-BRD9 construct. Finally, shown herein is also the degradation of endogenous BPTF and CECR2 through western blotting using the TP-238-based degraders in HEK293T cells.

### DESCRIPTION OF THE FIGURES

FIG. 1A is the chemical structures of previously reported non-BET bromodomain (BD) targeting degraders (bromodomain binding moiety in blue, E3 ligase ligand in green) and their biological activity.

FIG. 1B is a part of the bromodomain phylogenetic tree, showing class I, IV and II (BET) bromodomains

FIG. 1C is the chemical structures of pyridazinone and pyrimidine-based scaffolds with their affinity values for BPTF.

FIG. 2A is BPTF BRD with compounds 1 (yellow) and 2 (orange) overlays.

FIG. 2B is BPTF BRD with compound 1 (yellow) and TP-238 (green) overlay.

FIG. 2C is AlphaScreen binding isotherms with BPTF for pyridazinone-based degraders in Table 1, indicating bromodomain binding activity is retained.

FIG. 2D is AlphaScreen binding isotherms for TP-238 based degraders with BPTF.

FIG. 3A is a cartoon of an in vitro AlphaScreen ternary complex formation assay workflow.

FIG. 3B is a plot of ternary complex formation with BPTF.

FIG. 3C is a plot of ternary complex formation with BRD9.

FIG. 3D is a plot of ternary complex formation with CECR2.

FIG. 3E is a plot of ternary complex formation with PCAF bromodomains.

FIG. 4A is a cartoon of a NanoBRET assay format used to monitor ternary complex formation and degradation in HEK293T cells.

FIG. 4B is bar graphs of in-cell ternary complex formation rank-ordered by determining fold-change in BRET ratio compared to DMSO control.

FIG. 4C is bar graphs of degradation of the Nluc-BPTF-BD construct studied by measuring the total donor luminescence in the absence of MG-132. Degradation was rescued on pre-treatment with 10 μM MG-132.

FIG. 5A is bar graphs of in-cell NanoBRET experiments with full-length (FL) BRD9, indicating ternary complex formation.

FIG. 5B is bar graphs of degradation of the Nluc-BRD9-FL fusion construct.

FIG. 6A is Western blotting in HEK293T cells with 6 h treatment of compounds 8 and 9 with BPTF.

FIG. 6B is Western blotting in HEK293T cells with 6 h treatment of compound 9 with CECR2.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The disclosure generally relates to a compound of the Formula (I):

A-L¹-B Formula (I)

Or a pharmaceutically acceptable salt, polymorph, prodrug, solvate or clathrate thereof, wherein A is a ligand for the bromodomains of at least one of Bromodomain PHD Finger Transcription Factor (BPTF), CECR2, (Bromodomain Containing 9) BRD9, and PCAF/GCN5; B is an E3 ligase ligand; and L¹ is a linker.

A can be a group of the formula X—N(H)—Z, wherein L¹ is covalently attached to X, wherein:

Z is a group of the formula:

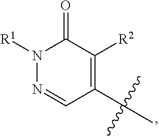

wherein R¹ is H or alkyl and R² is halo; and
X is a heterocyclyl group.

X can be a group of the formula:

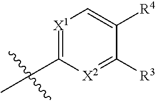

wherein X¹ and X² are each independently CH or N; and R² and R³, together with the carbon atoms to which they are attached, form a heterocyclyl group. For example, X can be a group of the formula:

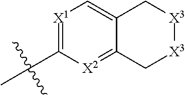

wherein X¹ and X² are each independently CH or N; and each X³ is, independently, CH₂ or N provided that one of X³ is N and X³ is attached to L¹. X can be a group of the formula:

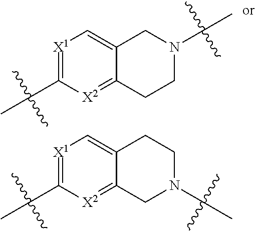

wherein X¹ and X² are each independently CH or N. In any of the foregoing examples of X groups, X¹ and X² can be CH simultaneously.

Examples of X—Y—Z groups include groups of the formula:

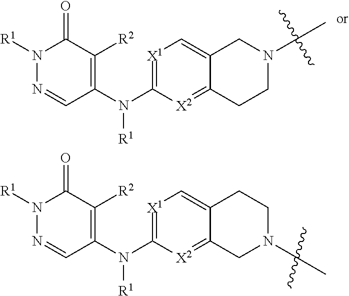

Alternatively, A can be a group of the formula X⁴—Z²—Y¹—Z¹, wherein L¹ is covalently attached to X⁴, wherein:

Z¹ is a five-membered heterocyclyl group;
Y¹ is alkyl-NR¹—, wherein R¹ is H or alkyl;
Y² is a six-membered heterocyclyl group; and
X⁴ is aryl-O—.

Examples of Z¹ groups include groups of the formula:

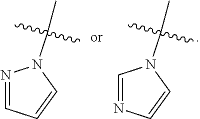 [SMILES: *n1cccn1 ; *n1ccnc1]

An example of a Z² group is a group of the formula:

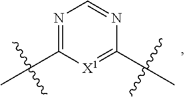

wherein X¹ is CH or N.

X⁴ can be a group of the formula:

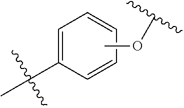

In any of the foregoing, L¹ can be acyl, amido, alkyl, alkenyl, alkynyl, heterocyclyl, and combinations thereof, optionally interrupted by one or more heteroatoms. Examples of the one or more heteroatoms include, but are not limited to, —O—, —NR¹— (wherein R¹ is H or alkyl), and —S(O)ₙ— (wherein n is 0, 1 or 2). Examples of L¹ groups include, but are not limited to, alkyl, -(alkyl-O)ₓ—, -(alkyl-NR¹)ₓ— (wherein x is an integer from 1 to 10), —NR¹—C(O)— (wherein R¹ is H or alkyl), a six-membered heterocyclyl group, and combinations thereof. Thus, for example, L¹ can be:

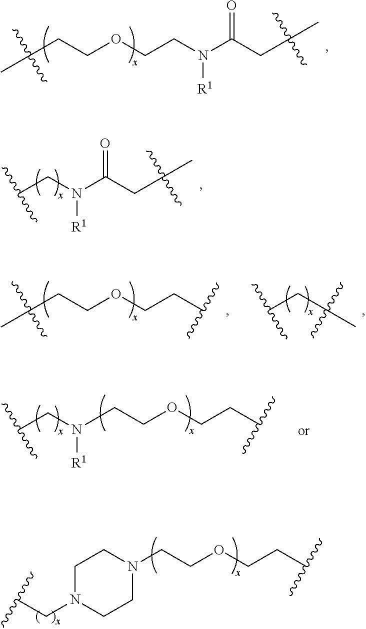

or combinations thereof, wherein R¹ is H or alkyl and each x is, independently, an integer from from 1 to 10.

In any of the foregoing, B can be a group of the formula:

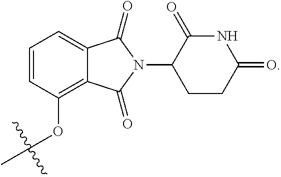 [SMILES: *Oc1cccc2c1C(=O)N(C1CCC(=O)NC1=O)C2=O]

Examples of compounds of the Formula (I) include compounds of the formula:

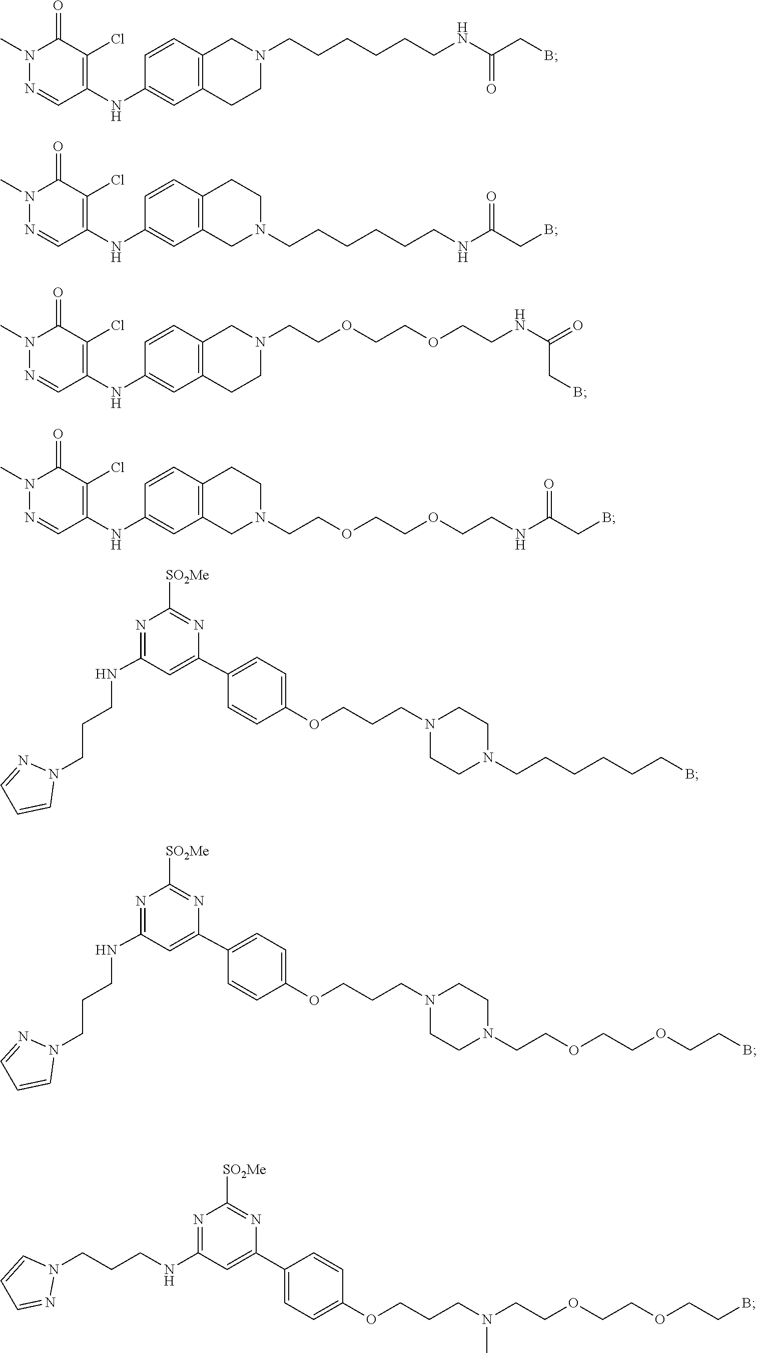

or a pharmaceutically acceptable salt, polymorph, prodrug, solvate or clathrate thereof. The group B, in any of the foregoing examples, can be a group of the formula:

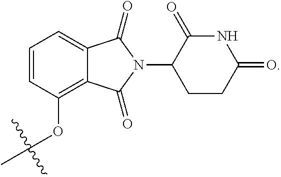 [SMILES: *Oc1cccc2c1C(=O)N(C1CCC(=O)NC1=O)C2=O]

The disclosure also provides a pharmaceutical composition comprising a compound of any of the preceding formulae and a pharmaceutically acceptable carrier. The disclosure also provides a pharmaceutical composition comprising a therapeutically effective amount of a compound of Formula (I), and a pharmaceutically acceptable carrier.

The disclosure also contemplates pharmaceutical compositions comprising one or more compounds of the various embodiments of the disclosure and one or more pharmaceutically acceptable excipients. A “pharmaceutical composition” refers to a chemical or biological composition suitable for administration to a subject (e.g., mammal). Such compositions can be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” is a carrier, sometimes a liquid, in which an active therapeutic agent is formulated. The excipient generally does not provide any pharmacological activity to the formulation, though it can provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes, but is not limited to, any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the disclosure is contemplated. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions of the disclosure can be orally administered as a capsule (hard or soft), tablet (film coated, enteric coated or uncoated), powder or granules (coated or uncoated) or liquid (solution or suspension). The formulations can be conveniently prepared by any of the methods well-known in the art. The pharmaceutical compositions of the disclosure can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments of the disclosure can vary according to factors such as the disease state, age, gender, weight, patient history, risk factors, predisposition to disease, administration route, pre-existing treatment regime (e.g., possible interactions with other medications), and weight of the subject. Dosage regimens can be adjusted to provide the optimum therapeutic response. For example, a single bolus can be administered, several divided doses can be administered over time, or the dose can be proportionally reduced or increased as indicated by the exigencies of therapeutic situation.

A “dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of sensitivity in subjects. In therapeutic use for treatment of conditions in mammals (e.g., humans) for which the compounds of the disclosure or an appropriate pharmaceutical composition thereof are effective, the compounds of the disclosure can be administered in an effective amount. The dosages as suitable for this disclosure can be a composition, a pharmaceutical composition or any other compositions described herein.

For each of the recited embodiments, the dosage is typically administered once, twice, or thrice a day, although more frequent dosing intervals are possible. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition, the dosage can be administered for as long as signs and/or symptoms persist. The patient can require “maintenance treatment” where the patient is receiving dosages every day for months, years, or the remainder of their lives. In addition, the composition of this disclosure can be to effect prophylaxis of recurring symptoms. For example, the dosage can be administered once or twice a day to prevent the onset of symptoms in patients at risk, especially for asymptomatic patients.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to, for example, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

As stated in the disclosure, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the disclosure being administered, the size of the subject, or the severity of the disease or condition. One of ordinary skill in the art can empirically determine the effective amount of a particular compound of the disclosure and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of a compound are, for human subjects, from about 0.01 milligrams/kg per day to 1000 milligrams/kg per day. Oral doses in the range of 0.5 to 50 milligrams/kg, in one or more administrations per day, can yield therapeutic results. Dosage may be adjusted appropriately to achieve desired drug levels, local or systemic, depending upon the mode of administration. For example, intravenous administration may vary from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

For clinical use, any compound of the disclosure can be administered in an amount equal or equivalent to 0.2-2000 milligram (mg) of compound per kilogram (kg) of body weight of the subject per day. The compounds of the disclosure can be administered in a dose equal or equivalent to 2-2000 mg of compound per kg body weight of the subject per day. The compounds of the disclosure can be administered in a dose equal or equivalent to 20-2000 mg of compound per kg body weight of the subject per day. The compounds of the disclosure can be administered in a dose equal or equivalent to 50-2000 mg of compound per kg body weight of the subject per day. The compounds of the disclosure can be administered in a dose equal or equivalent to 100-2000 mg of compound per kg body weight of the subject per day. The compounds of the disclosure can be administered in a dose equal or equivalent to 200-2000 mg of compound per kg body weight of the subject per day. Where a precursor or prodrug of the compounds of the disclosure is to be administered rather than the compound itself, it is administered in an amount that is equivalent to, i.e., sufficient to deliver, the above-stated amounts of the compounds of the invention.

The formulations of the compounds of the disclosure can be administered to human subjects in therapeutically effective amounts. Typical dose ranges are from about 0.01 microgram/kg to about 2 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments may be used to optimize the dose and dosing frequency for any particular compound.

The compounds of the disclosure can be administered at a concentration in the range from about 0.001 microgram/kg to greater than about 500 mg/kg. For example, the concentration may be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed by the present invention.

The compounds of the disclosure can be administered at a dosage in the range from about 0.2 milligram/kg/day to greater than about 100 mg/kg/day. For example, the dosage may be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The compounds of the disclosure can be administered at a dosage in the range from about 0.25 milligram/kg/day to about 25 mg/kg/day. For example, the dosage may be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

In various embodiments, the compound or precursor thereof is administered in concentrations that range from 0.01 micromolar to greater than or equal to 500 micromolar. For example, the dose may be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed by the present invention.

In various embodiments, the compound or precursor thereof is administered at concentrations that range from 0.10 microgram/mL to 500.0 microgram/mL. For example, the concentration may be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed by the present invention.

The disclosure also provides a method of modulating the activity (e.g., inhibit the activity or degrade) of at least one of BPTF, CECR2, BRD9, and PCAF/GCN5 comprising administering a therapeutically effective amount of a compound of any preceding claim, or a pharmaceutical composition comprising said compound, to a subject in need thereof.

The disclosure also provides a method for treating cancer comprising administering a therapeutically effective amount of one or more compounds of any preceding claim, or a pharmaceutical composition comprising said compound, to a subject in need thereof. Examples of cancers include, but are not limited to breast cancer, non-small-cell lung cancer, colorectal cancer or high-grade gliomas.

The compounds described herein can be administered as the sole therapeutics agents or along with at least one chemotherapeutic agent in combination with the at least one compound of any preceding claim. Examples of chemotherapeutic agents include, but are not limited to chemotherapeutic agents such as abiraterone acetate, alemtuzumab, altretamine, belinostat, bevacizumab, blinatumomab, bleomycin, bortezomib, brentuximab, vedotin, busulfan, cabazitaxel, capecitabine, carboplatin, carmustine, ceritinib, cetuximab, chlorambucil, cisplatin, cladribine, crizotinib, cyclophosphamide, cytarabine, dabrafenib, dacarbazine, dactinomycin dasatinib, daunorubicin, daunoXome, depoCytd docetaxel, doxil I, doxorubicin, epirubicin, eribulin mesylate, erlotinib, estramustine, etoposide, everolimus, floxuridine, fludarabine, fluorouracil, gefitinib, gemcitabine, gliadel wafers, hydroxyurea, ibritumomab, ibrutinib, idarubicin, idelalisib, ifosfamide, imatinib, ipilimumab, irinotecan, ixabepilone, lanreotide, lapatinib, lenalidomide, lenvatinib, lomustine, mechlorethamine, melphalan, mercaptopurine, methotrexate, mitomycin, mitoxantrone, nilotinib, nivolumab, ofatumumab, olaparib, oxaliplatin, paclitaxel, palbociclib, panitumumab, pazopanib, panobinostat, PEG-asparaginase, peginterferon alfa-2b, pembrolizumab, pemetrexed, pentostatin, pralatrexate, procarbazine, ramucirumab, rituximab, romidepsin, ruxolitinib, sipuleucel-T, sorafenib, streptozocin, sunitinib, temozolomide, temsirolimus, teniposide, thalidomide, thioguanine, thiotepa, topotecan, tositumomab, trametinib, trastuzumab, valrubicin, vandetanib, vemurafenib, vinblastine, vincristine, vinorelbine, and the like. The compounds described herein can be administered as the sole therapeutics agents or along with anti-inflammatory agents, anti-viral agents, antibacterial agents, antimicrobial agents, immunomodulatory drugs, such as lenalidomide, pomalidomide or thalidomide, histone deacetylase inhibitors, such as panobinostat, preservatives or combinations thereof.

The disclosure also provides a method for treating inflammation comprising administering a therapeutically effective amount of one or more compounds of any preceding claim, or a pharmaceutical composition comprising said compound, to a subject in need thereof.

The term “therapeutically effective amount” as used herein, refers to that amount of one or more compounds of the various examples of the disclosure that elicits a biological or medicinal response in a tissue system, animal or human, that is being sought by a researcher, veterinarian, medical doctor or other clinician, which includes alleviation of the symptoms of the disease or disorder being treated. In some examples, the therapeutically effective amount is that which can treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

The term “alkyl” as used herein refers to substituted or unsubstituted straight chain, branched and cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopently, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted from a set of groups including amino, hydroxy, cyano, carboxy, nitro, thio and alkoxy, but not including halogen groups. Thus, in some embodiments alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl.

The term “alkenyl” as used herein refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl.

For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C═CH, —C═C(CH₃), —C═C(CH₂CH₃), —CH₂C═CH, —CH₂C═C(CH₃), and —CH₂C═C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

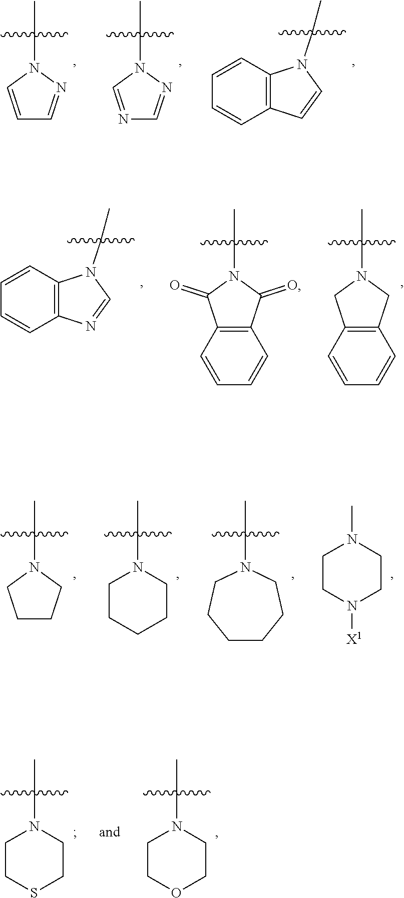

wherein X¹ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” or “halide” group, as used herein, by themselves or as part of another substituent, mean, unless otherwise stated, a fluorine, chlorine, bromine, or iodine atom.

The term “amino” as used herein refers to a substituent of the form —NH₂, —NHR, —NR₂, —NR₃+, wherein each R is independently selected, and protonated forms of each, except for —NR₃+, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like.

The term “formyl” as used herein refers to a group containing a carbonyl moiety wherein the group is bonded via the carbonyl carbon atom. The carbonyl carbon atom is also bonded to a hydrogen atom.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “arylcarbonyl” as used herein refers to a group containing a carbonyl moiety wherein the group is bonded via the carbonyl carbon atom. The carbonyl carbon atom is also bonded to an aryl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to an nitrogen atom which is bonded to one or more aryl group instead of, or in addition to, the one or more alkyl group. In a further case, which is also an alkylamido as the term is defined herein, the carbonyl carbon atom is bonded to an nitrogen atom which is bonded to one or more alkenyl group instead of, or in addition to, the one or more alkyl and or/aryl group. In a further case, which is also an alkylamido as the term is defined herein, the carbonyl carbon atom is bonded to an nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “alkylthio” as used herein refers to a sulfur atom connected to an alkyl, alkenyl, or alkynyl group as defined herein.

The term “arylthio” as used herein refers to a sulfur atom connected to an aryl group as defined herein.

The term “alkylsulfonyl” as used herein refers to a sulfonyl group connected to an alkyl, alkenyl, or alkynyl group as defined herein.

The term “alkylsulfinyl” as used herein refers to a sulfinyl group connected to an alkyl, alkenyl, or alkynyl group as defined herein.

The term “dialkylaminosulfonyl” as used herein refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups in place of the alkyl groups.

The term “dialkylamino” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups.

The term “dialkylamido” as used herein refers to an amido group connected to two alkyl groups, as defined herein, and which can optionally be linked together to form a ring with the nitrogen. This term also includes the group where the nitrogen is further connected to one or two alkenyl groups in place of the alkyl groups.

The term “substituted” as used herein refers to a group that is substituted with one or more groups including, but not limited to, the following groups: halogen (e.g., F, Cl, Br, and I), R, OR, OC(O)N(R)₂, CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl or (C₆-C₂₀)aryl. Substituted also includes a group that is substituted with one or more groups including, but not limited to, the following groups: fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

In some instances, the compounds described herein (e.g., the compounds of the Formula (I)) can contain chiral centers. All diastereomers of the compounds described herein are contemplated herein, as well as racemates.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the disclosure. Examples of prodrugs include, but are not limited to, derivatives and metabolites of a compound of the disclosure that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

As used herein, the term “subject” or “patient” refers to any organism to which a composition described herein can be administered, e.g., for experimental, diagnostic, prophylactic and/or therapeutic purposes. Subject refers to a mammal receiving the compositions disclosed herein or subject to disclosed methods. It is understood and herein contemplated that “mammal” includes but is not limited to humans, non-human primates, cows, horses, dogs, cats, mice, rats, rabbits, and guinea pigs.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the disclosure, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

Each embodiment described above is envisaged to be applicable in each combination with other embodiments described herein. For example, embodiments corresponding to Formula (I) are equally envisaged as being applicable to other compounds.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed can be resorted to by those of ordinary skill in the art, and that such modifications and variations are considered to be within the scope of embodiments of the present disclosure

### EXAMPLES

The disclosure can be better understood by reference to the following examples which are offered by way of illustration. The disclosure is not limited to the examples given herein.

Materials and Methods. All commercially available reagents were used without further purification. Flash column chromatography was performed on a Teledyne-Isco Rf-plus CombiFlash instrument with RediSep columns. NMR spectra were collected on a Bruker Avance III AX-400 or a Bruker Avance III HD-500 equipped with a Prodigy TCI cryoprobe. Chemical shifts (δ) were reported in parts per million (ppm) and referenced to residual solvent signals for Chloroform-d (¹H 7.26 ppm), Dimethyl Sulfoxide-d₆ (¹H 2.50 ppm, ¹³C 39.5 ppm) and Methanol-d₄ (¹H 3.31 ppm, ¹³C 49.0 ppm). Coupling constants (J) are in Hz. Splitting patterns were reported as s (singlet), d (doublet), t (triplet), q (quartet) and m (multiplet). High resolution ESI-MS spectra were recorded on a Thermo Fischer Orbitrap Velos equipped with an autosampler. Where stated, compounds were purified by reverse-phase high-performance liquid chromatography (RP-HPLC) on a C-18 column using 0.1% TFA water and CH₃CN as solvents and TFA salts were quantified using the procedure described by Carlson et. al.³⁶

Purity Analysis. All compounds tested in cells were ≥95% pure by RP-HPLC. Compounds 3-9 were run on a RP-HPLC with a C-18 column. A gradient of 0-40% ACN in 0.1% TFA H₂O over 60 min was used for compounds 3, 5 and 6, and 0-60% ACN in 0.1% TFA H₂O over 60 min for compound 4. Gradients for compounds 8-10.

Synthetic methods. The synthesis and characterization of compounds 1 and 2 were described previously.

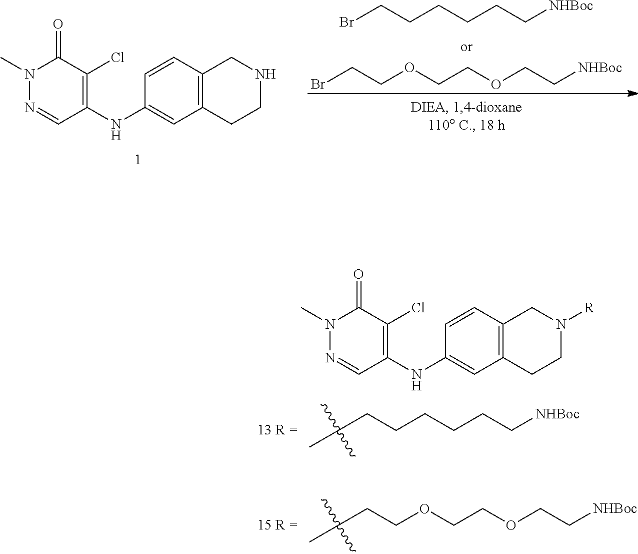

General procedure A for the synthesis of intermediates 13-16. Compound 1 or 2 (1.0 eq.) was stirred in 1,4-dioxane at room temperature, followed by addition of the N-Boc linker (1.1 eq.) and N,N-Diisopropylethylamine (1.5 eq.). The reaction mixture was heated in a sealed tube at 110° C. for 18 h. Following completion of the reaction, the 1,4-dioxane was removed by rotary evaporation. The crude mixture was extracted into ethyl acetate, washed with a saturated sodium bicarbonate solution (3×20 mL) and finally with brine (20 mL). The organic layer was dried over magnesium sulfate, filtered, concentrated in vacuo and purified by flash column chromatography (CombiFlash Rf system: 4 g silica, DCM/methanol, 0-20% methanol, 30 minutes).

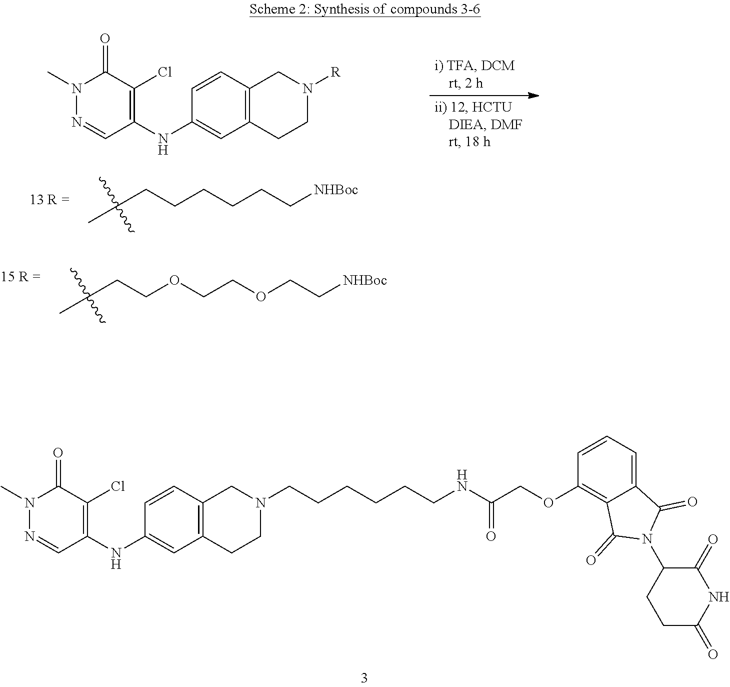

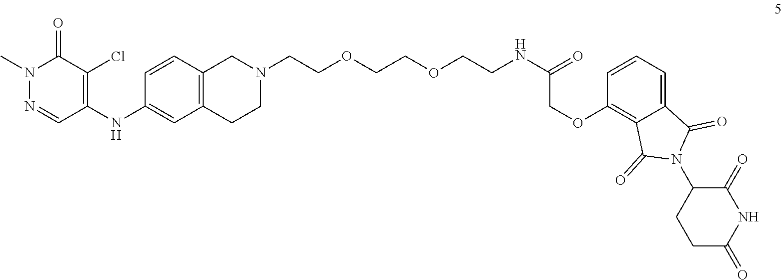 [SMILES: Cn1ncc(Nc2ccc3c(c2)CCN(CCOCCOCCNC(=O)COc2cccc4c2C(=O)N(C2CCC(=O)NC2=O)C4=O)C3)c(Cl)c1=O]

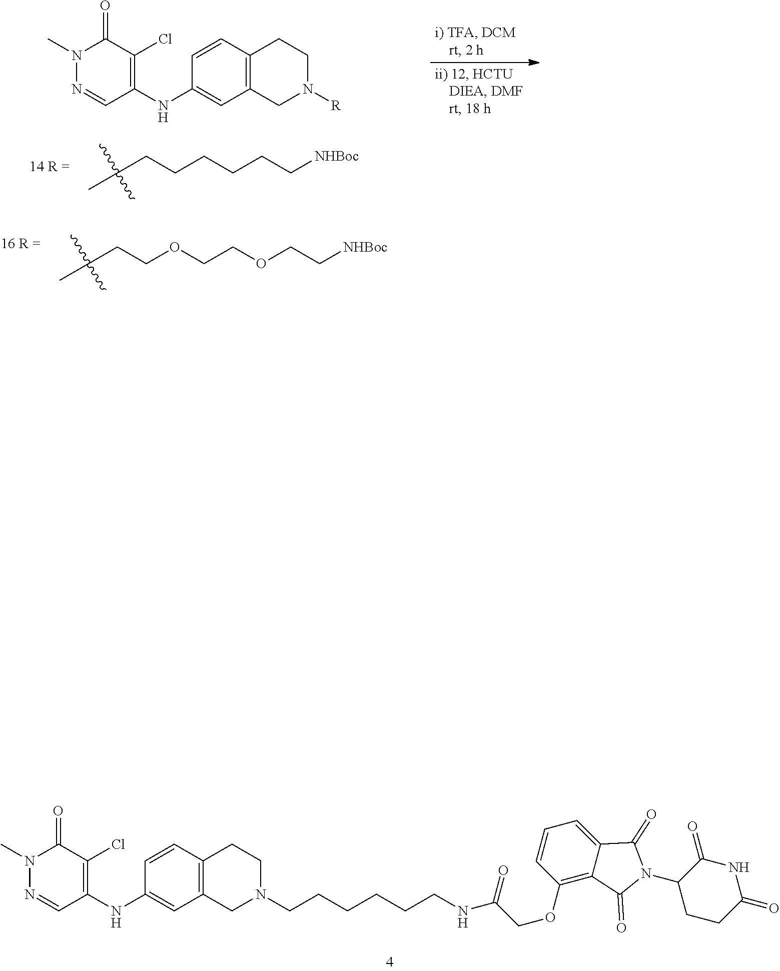

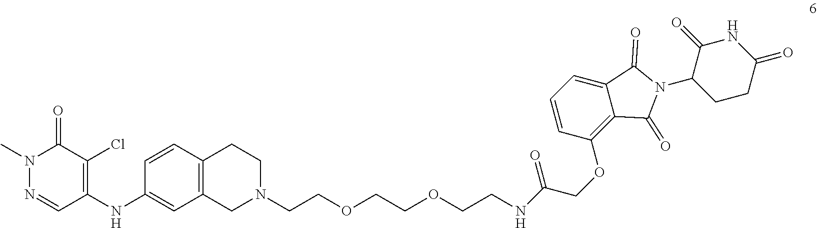 [SMILES: Cn1ncc(Nc2ccc3c(c2)CN(CCOCCOCCNC(=O)COc2cccc4c2C(=O)N(C2CCC(=O)NC2=O)C4=O)CC3)c(Cl)c1=O]

General procedure B for the synthesis of compounds 3-6. Step 1: Compounds 13-16 were stirred in DCM at room temperature, followed by addition of trifluoroacetic acid (5.0 eq.) and stirred at room temperature for an additional 2 h. Following completion of the reaction, the mixture was blown dry under a stream of nitrogen and the crude product was used without further purification.

Step 2: The crude product from Step 1 (1.0 eq.) was stirred in dry DMF and DIEA (4.0 eq.) at room temperature. A mixture of 12 (1.2 eq.) and HCTU (1.2 eq.) in dry DMF was then added and the reaction mixture was stirred at room temperature for 16 h. A portion of the crude material was then purified by reverse-phase HPLC.

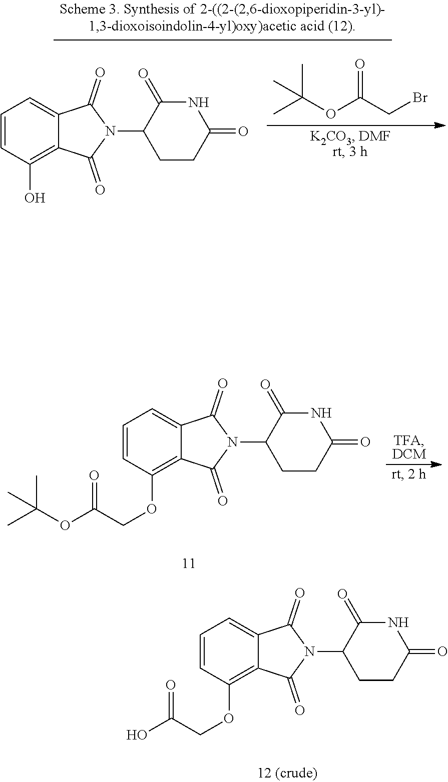

tert-butyl 2-((2-(2,6-dioxopiperidin-3-yl)-1,3-dioxoisoindolin-4-yl)oxy)acetate (11). Compound 11 was synthesized according to literature procedure.³⁷ 2-(2,6-dioxopiperidin-3-yl)-4-hydroxyisoindoline-1,3-dione (240 mg, 0.88 mmol, 1.0 eq.) was dissolved in DMF (2 mL) at room temperature, followed by the addition of potassium carbonate (180 mg, 1.30 mmol, 1.5 eq.) and tert-butyl 2-bromoacetate (186 mg, 0.95 mmol, 1.1 eq.). The reaction mixture was stirred at room temperature. After 2 h, the mixture was extracted into ethyl acetate, washed with water (20 mL) and finally with brine (20 mL). The organic layer was dried over magnesium sulfate, filtered, concentrated in vacuo and purified by flash column chromatography (CombiFlash Rf system: 24 g silica, hexanes/ethyl acetate, 0-100% ethyl acetate, 16 minutes) to obtain a white solid (198 mg, 58% yield). ¹H NMR (500 MHz, DMSO) δ 11.11 (s, 1H), 7.80 (dd, J=8.5, 7.2 Hz, 1H), 7.48 (d, J=6.9 Hz, 1H), 7.38 (d, J=8.1 Hz, 1H), 5.10 (dd, J=12.8, 5.5 Hz, 1H), 4.97 (s, 2H), 2.89 (ddd, J=16.9, 13.8, 5.5 Hz, 1H), 2.63-2.56 (m, 1H), 2.55-2.51 (m, 1H), 2.09-2.00 (m, 1H), 1.43 (s, 9H), H₂O from solvent at 3.32 ppm, ethyl acetate impurity at 4.02 ppm, 1.99 ppm and 1.17 ppm (4.8% by weight).

2-((2-(2,6-dioxopiperidin-3-yl)-1,3-dioxoisoindolin-4-yl)oxy)acetic acid (12). Compound 11 was stirred in DCM at room temperature, followed by addition of trifluoroacetic acid (5.0 eq.) and stirred at room temperature for an additional 2 h. Following completion of the reaction, the mixture was blown dry under a stream of nitrogen and the crude product 12 was used without further purification.

tert-butyl (5-(6-((5-chloro-1-methyl-6-oxo-1,6-dihydropyridazin-4-yl)amino)-3,4-dihydroisoquinolin-2(1H)-yl)pentyl)carbamate (13). Following the general procedure A, (1 (63 mg, 0.22 mmol, 1.0 eq.), tert-butyl (6-bromohexyl)carbamate (67 mg, 0.24 mmol, 1.1 eq.), N,N-Diisopropylethylamine (57 μL, 0.33 mmol, 1.5 eq.), 1,4-dioxane (1 mL)), product 13 was obtained as a brown solid (42 mg, 40% yield). ¹H NMR (500 MHz, DMSO) δ 8.61 (s, 1H), 7.61 (s, 1H), 7.08 (d, J=8.1 Hz, 1H), 6.99 (dd, J=8.1, 2.3 Hz, 1H), 6.97 (d, J=2.2 Hz, 1H), 6.76 (t, J=5.8 Hz, 1H), 3.60 (s, 3H), 3.51 (s, 2H), 2.89 (q, J=6.6 Hz, 2H), 2.79 (t, J=5.9 Hz, 2H), 2.62 (t, J=5.9 Hz, 2H), 2.42 (t, J=7.3 Hz, 2H), 1.50 (q, J=7.2 Hz, 2H), 1.37 (s, 9H), 1.33-1.21 (m, 4H), H₂O from solvent at 3.32 ppm, DCM impurity at 5.76 ppm (2.5% by weight). ¹³C NMR (126 MHz, DMSO) δ 157.0, 155.6, 142.6, 136.1, 135.5, 132.1, 127.5, 127.3, 123.6, 121.3, 107.8, 77.3, 57.7, 55.2, 50.4, 29.5, 28.3, 26.6, 26.5, 26.2 (one resonance obscured by solvent, two resonances overlapping). HRMS (ESI-TOF) calculated for C₂₅H₃₇ClN₅O₃⁺ [M+H]⁺: 490.2579, observed 490.2546.

tert-butyl (6-(7-((5-chloro-1-methyl-6-oxo-1,6-dihydropyridazin-4-yl)amino)-3,4-dihydroisoquinolin-2(1H)-yl)hexyl)carbamate (14). Following the general procedure A, (2 (60 mg, 0.21 mmol, 1.0 eq.), tert-butyl (6-bromohexyl)carbamate (64 mg, 0.23 mmol, 1.1 eq.), N,N-Diisopropylethylamine (54 μL, 0.31 mmol, 1.5 eq.), 1,4-dioxane (1 mL)), product 14 was obtained as a yellow solid (28 mg, 26% yield). ¹H NMR (500 MHz, DMSO) δ 8.61 (s, 1H), 7.60 (s, 1H), 7.11 (d, J=8.1 Hz, 1H), 7.00 (dd, J=8.1, 2.3 Hz, 1H), 6.93 (d, J=2.3 Hz, 1H), 6.75 (t, J=5.8 Hz, 1H), 3.60 (s, 3H), 3.51 (s, 2H), 2.89 (q, J=6.6 Hz, 2H), 2.78 (t, J=5.9 Hz, 2H), 2.63 (t, J=5.8 Hz, 2H), 2.41 (t, J=7.3 Hz, 2H), 1.50 (t, J=7.2 Hz, 2H), 1.36 (s, 9H), 1.27 (m, 4H), H₂O from solvent at 3.32 ppm. ¹³C NMR (126 MHz, DMSO) δ 157.0, 155.6, 142.6, 136.2, 135.7, 131.4, 129.3, 127.5, 121.9, 121.7, 107.7, 77.3, 57.6, 55.4, 50.5, 29.5, 28.3, 26.6, 26.5, 26.2 (one resonance obscured by solvent, two resonances overlapping). HRMS (ESI-TOF) calculated for C₂₅H₃₇ClN₅O₃⁺ [M+H]⁺: 490.2579, observed 490.2547.

tert-butyl (2-(2-(2-(6-((5-chloro-1-methyl-6-oxo-1,6-dihydropyridazin-4-yl)amino)-3,4-dihydroisoquinolin-2(1H)-yl)ethoxy)ethoxy)ethyl)carbamate (15). Following the general procedure A, (1 (60 mg, 0.21 mmol, 1.0 eq.), tert-butyl (2-(2-(2-bromoethoxy)ethoxy)ethyl)carbamate (71 mg, 0.23 mmol, 1.1 eq.), N,N-Diisopropylethylamine (54 μL, 0.31 mmol, 1.5 eq.), 1,4-dioxane (1 mL)), product 15 was obtained as a brown solid (53 mg, 49% yield). ¹H NMR (500 MHz, DMSO) δ 8.62 (s, 1H), 7.60 (s, 1H), 7.06 (d, J=8.1 Hz, 1H), 6.99 (dd, J=8.1, 2.3 Hz, 1H), 6.97 (d, J=2.1 Hz, 1H), 6.75 (t, J=5.8 Hz, 1H), 3.61-3.58 (m, 7H, overlapping resonances), 2.73-2.68 (m, 4H), 3.38 (t, J=6.2 Hz, 2H), 3.06 (q, J=6.1 Hz, 2H), 2.79 (t, J=5.9 Hz, 2H), 2.70 (t, J=5.8 Hz, 2H), 2.64 (t, J=5.9 Hz, 2H), 1.37 (s, 9H), H₂O from solvent at 3.32 ppm. ¹³C NMR (126 MHz, DMSO) δ 157.0, 155.6, 142.6, 136.1, 135.3, 132.1, 127.5, 127.3, 123.6, 121.4, 107.8, 77.6, 69.7, 69.5, 69.2, 68.5, 57.0, 55.4, 50.7, 28.7, 28.2 (one resonance obscured by solvent, two resonances overlapping). HRMS (ESI-TOF) calculated for C₂₅H₃₇ClN₅O₅⁺ [M+H]⁺: 522.2478, observed 522.2444.

tert-butyl (2-(2-(2-(7-((5-chloro-1-methyl-6-oxo-1,6-dihydropyridazin-4-yl)amino)-3,4-dihydroisoquinolin-2(1H)-yl)ethoxy)ethoxy)ethyl)carbamate (16). Following the general procedure A, (2 (75 mg, 0.26 mmol, 1.0 eq.), tert-butyl (2-(2-(2-bromoethoxy)ethoxy)ethyl)carbamate (89 mg, 0.28 mmol, 1.1 eq.), N,N-Diisopropylethylamine (67 μL, 1.5 mmol, 1.5 eq.), 1,4-dioxane (1 mL)), product 16 was obtained as a brown solid (39 mg, 36% yield). ¹H NMR (500 MHz, DMSO) δ 8.61 (s, 1H), 7.60 (s, 1H), 7.11 (d, J=8.2 Hz, 1H), 7.00 (dd, J=8.1, 2.3 Hz, 1H), 6.92 (d, J=2.3 Hz, 1H), 6.74 (t, J=5.8 Hz, 1H), 3.63-3.56 (m, 7H, overlapping resonances), 3.53-3.49 (m, 4H), 3.38 (t, J=6.1 Hz, 2H), 3.05 (q, J=6.0 Hz, 2H), 2.78 (t, J=5.7 Hz, 2H), 2.71 (t, J=5.8 Hz, 2H), 2.64 (t, J=5.9 Hz, 2H), 1.36 (s, 9H), H₂O from solvent at 3.32 ppm, DCM impurity at 5.75 ppm (2.4% by weight). ¹³C NMR (126 MHz, DMSO) δ 157.0, 155.6, 142.6, 136.1, 135.7, 131.2, 129.3, 127.5, 121.9, 121.7, 107.7, 77.6, 69.7, 69.5, 69.2, 68.5, 56.9, 55.6, 50.8, 28.2 (overlapping resonances), (one resonance obscured by solvent, two resonances overlapping). HRMS (ESI-TOF) calculated for C₂₅H₃₇ClN₅O₅⁺ [M+H]⁺: 522.2478, observed 522.2442.

N-(5-(6-((5-chloro-1-methyl-6-oxo-1,6-dihydropyridazin-4-yl)amino)-3,4-dihydroisoquinolin-2(1H)-yl)pentyl)-2-((2-(2,6-dioxopiperidin-3-yl)-1,3-dioxoisoindolin-4-yl)oxy)acetamide (3). Following the general procedure B, compound 13 was Boc deprotected in Step 1 and the crude material was used for Step 2: (29 mg, 0.07 mmol, 1.0 eq.), 11 (30 mg, 0.09 mmol, 1.2 eq.), HCTU (37 mg, 0.09 mmol, 1.2 eq.), N,N-Diisopropylethylamine (52 μL, 0.30 mmol, 4.0 eq.), DMF (0.5 mL). A portion of the crude product was purified by reverse-phase semi-prep HPLC (5-30% ACN in 0.1% TFA water over 25 minutes, C18 column) to obtain product 3 as a white solid (2×TFA salt). ¹H NMR (500 MHz, DMSO) δ 11.12 (s, 1H), 9.76 (s, 1H), 8.79 (s, 1H), 8.02-7.92 (m, 1H), 7.82 (dd, J=8.5, 7.3 Hz, 1H), 7.66 (s, 1H), 7.51 (d, J=7.2 Hz, 1H), 7.41 (d, J=8.5 Hz, 1H), 7.23 (d, J=8.3 Hz, 1H), 7.16 (dd, J=8.3, 2.3 Hz, 1H), 7.13 (d, J=2.2 Hz, 1H), 5.12 (dd, J=12.9, 5.4 Hz, 1H), 4.78 (s, 2H), 4.57-4.51 (m, 1H), 4.26 (dd, J=15.5, 8.1 Hz, 1H), 3.75-3.67 (m, 1H), 3.62 (s, 3H), 3.36-3.24 (m, 1H), 3.24-3.13 (m, 4H), 3.13-2.97 (m, 2H), 2.95-2.81 (m, 1H), 2.65-2.56 (m, 1H), 2.54 (d, J=4.4 Hz, 1H), 2.09-1.99 (m, 1H), 1.78-1.67 (m, 2H), 1.58-1.43 (m, 2H), 1.39-1.27 (m, 4H). ¹³C NMR (126 MHz, DMSO) δ 172.8, 169.9, 166.7, 165.5, 157.0, 155.0, 142.0, 138.1, 136.9, 133.0, 132.5, 127.7, 124.7, 122.6, 121.8, 120.4, 116.8, 116.1, 109.2, 67.7, 55.1, 51.8, 48.8, 38.1, 30.9, 28.8, 25.7, 25.6, 25.0, 23.4, 22.0 (one resonance obscured by solvent, three resonances overlapping). HRMS (ESI-TOF) calculated for C₃₅H₃₉ClN₇O₇⁺ [M+H]⁺: 704.2594, observed 704.2552.

N-(6-(7-((5-chloro-1-methyl-6-oxo-1,6-dihydropyridazin-4-yl)amino)-3,4-dihydroisoquinolin-2(1H)-yl)hexyl)-2-((2-(2,6-dioxopiperidin-3-yl)-1,3-dioxoisoindolin-4-yl)oxy)acetamide (4). Following the general procedure B, compound 14 was boc deprotected in Step 1 and the crude material was used for Step 2: (13 mg, 0.03 mmol, 1.0 eq.), 11 (13 mg, 0.04 mmol, 1.2 eq.), HCTU (17 mg, 0.04 mmol, 1.2 eq.), N,N-Diisopropylethylamine (23 μL, 0.13 mmol, 4.0 eq.), DMF (0.5 mL). A portion of the crude product was purified by reverse-phase semi-prep HPLC (5-40% ACN in 0.1% TFA water over 25 minutes, C18 column) to obtain product 4 as a white solid (2×TFA salt). ¹H NMR (500 MHz, DMSO) δ 11.12 (s, 1H), 9.84 (s, 1H), 8.79 (s, 1H), 7.97 (t, J=5.5 Hz, 1H), 7.82 (dd, J=8.5, 7.3 Hz, 1H), 7.64 (s, 1H), 7.51 (d, J=7.2 Hz, 1H), 7.40 (d, J=8.5 Hz, 1H), 7.28 (d, J=8.3 Hz, 1H), 7.18 (dd, J=8.2, 2.3 Hz, 1H), 7.08 (d, J=2.3 Hz, 1H), 5.12 (dd, J=12.9, 5.4 Hz, 1H), 4.78 (s, 2H), 4.63-4.49 (m, 1H), 4.27 (dd, J=15.7, 8.0 Hz, 1H), 3.72 (d, J=12.4 Hz, 1H), 3.62 (s, 3H), 3.35-3.25 (m, 1H), 3.24-3.12 (m, 4H), 3.12-2.99 (m, 1H), 2.96-2.83 (m, 1H), 2.68-2.55 (m, 1H), 2.54 (d, J=4.5 Hz, OH), 2.10-1.95 (m, 1H), 1.81-1.66 (m, 2H), 1.55-1.40 (m, 2H), 1.36-1.22 (m, 4H). ¹³C NMR (126 MHz, DMSO) δ 172.8, 169.9, 166.7, 166.7, 165.5, 158.1, 157.0, 155.0, 142.1, 137.1, 136.9, 133.0, 129.6, 129.5, 127.7, 123.1, 120.9, 120.4, 116.8, 116.1, 109.0, 67.7, 55.1, 51.8, 49.0, 48.8, 38.1, 30.9, 28.8, 25.8, 25.6, 24.5, 23.4, 22.0 (one resonance obscured by solvent). HRMS (ESI-TOF) calculated for C₃₅H₃₈ClN₇O₉⁺ [M+H]⁺: 735.2420, observed 704.2554.

N-(2-(2-(2-(6-((5-chloro-1-methyl-6-oxo-1,6-dihydropyridazin-4-yl)amino)-3,4-dihydroisoquinolin-2(1H)-yl)ethoxy)ethoxy)ethyl)-2-((2-(2,6-dioxopiperidin-3-yl)-1,3-dioxoisoindolin-4-yl)oxy)acetamide (5). Following the general procedure B, compound 15 was Boc deprotected in Step 1 and the crude material was used for Step 2: (27 mg, 0.06 mmol, 1.0 eq.), 11 (25 mg, 0.08 mmol, 1.2 eq.), HCTU (32 mg, 0.08 mmol, 1.2 eq.), N,N-Diisopropylethylamine (45 μL, 0.26 mmol, 4.0 eq.), DMF (0.5 mL). A portion of the crude product was purified by reverse-phase semi-prep HPLC (5-30% ACN in 0.1% TFA water over 25 minutes, C18 column) to obtain product 5 as a white solid (1×TFA salt). ¹H NMR (500 MHz, DMSO) δ 11.13 (s, 1H), 10.10 (s, 1H), 8.77 (s, 1H), 8.00 (t, J=5.7 Hz, 1H), 7.81 (dd, J=8.5, 7.3 Hz, 1H), 7.65 (s, 1H), 7.50 (d, J=7.2 Hz, 1H), 7.41 (d, J=8.6 Hz, 1H), 7.21 (d, J=8.3 Hz, 1H), 7.14 (dd, J=8.3, 2.2 Hz, 1H), 7.11 (d, J=2.2 Hz, 1H), 5.11 (dd, J=12.9, 5.4 Hz, 1H), 4.79 (s, 2H), 4.53 (d, J=15.4 Hz, 1H), 4.34 (dd, J=15.6, 7.3 Hz, 1H), 3.84 (t, J=5.0 Hz, 2H), 3.77-3.70 (m, 1H), 3.64-3.62 (m, 2H), 3.61 (s, 3H), 3.61-3.58 (m, 2H), 3.50 (t, J=5.8 Hz, 2H), 3.46-3.37 (m, 3H), 3.34 (q, J=5.8 Hz, 2H), 3.20-3.09 (m, 1H), 3.09-2.99 (m, 1H), 2.95-2.84 (m, 1H), 2.60 (s, 1H), 2.57-2.52 (m, 1H), 2.08-1.99 (m, 1H). ¹³C NMR (126 MHz, DMSO) δ 172.8, 169.9, 166.9, 166.7, 165.5, 157.0, 154.9, 142.0, 138.1, 137.0, 133.0, 132.4, 127.7, 127.7, 124.5, 122.6, 121.7, 120.4, 116.8, 116.1, 109.2, 69.6, 69.4, 68.8, 67.5, 64.4, 54.3, 52.1, 49.0, 48.8, 38.4, 30.9, 24.7, 22.0 (one resonance obscured by solvent). HRMS (ESI-TOF) calculated for C₃₅H₃₉ClN₇O₉⁺ [M+H]⁺: 736.2492, observed 736.2445.

N-(2-(2-(2-(7-((5-chloro-1-methyl-6-oxo-1,6-dihydropyridazin-4-yl)amino)-3,4-dihydroisoquinolin-2(1H)-yl)ethoxy)ethoxy)ethyl)-2-((2-(2,6-dioxopiperidin-3-yl)-1,3-dioxoisoindolin-4-yl)oxy)acetamide (6). Following the general procedure B, compound 16 was Boc deprotected in Step 1 and the crude material was used for Step 2: (27 mg, 0.06 mmol, 1.0 eq.), 11 (26 mg, 0.08 mmol, 1.2 eq.), HCTU (32 mg, 0.08 mmol, 1.2 eq.), N,N-Diisopropylethylamine (45 μL, 0.26 mmol, 4.0 eq.), DMF (0.5 mL). A portion of the crude product was purified by reverse-phase semi-prep HPLC (5-30% ACN in 0.1% TFA water over 25 minutes, C18 column) to obtain product 6 as a white solid (2×TFA salt). ¹H NMR (500 MHz, DMSO) δ 11.13 (s, 1H), 9.99 (s, 1H), 7.97 (q, J=5.7 Hz, 1H), 7.81 (dd, J=8.5, 7.3 Hz, 1H), 7.62 (s, 1H), 7.50 (d, J=7.2 Hz, 1H), 7.40 (d, J=8.5 Hz, 1H), 7.26 (d, J=8.3 Hz, 1H), 7.17 (dd, J=8.2, 2.3 Hz, 1H), 7.08 (d, J=2.3 Hz, 1H), 5.11 (dd, J=12.9, 5.4 Hz, 1H), 4.77 (s, 2H), 4.53 (d, J=15.7 Hz, 1H), 4.40-4.31 (m, 1H), 3.82 (t, J=5.0 Hz, 2H), 3.79-3.71 (m, 1H), 3.67-3.55 (m, 7H), 3.48 (t, J=5.8 Hz, 2H), 3.45-3.36 (m, 3H), 3.33 (q, J=5.5 Hz, 2H), 3.18-3.08 (m, 1H), 3.08-2.99 (m, 1H), 2.95-2.84 (m, 1H), 2.64-2.56 (m, 1H), 2.57-2.52 (m, 1H), 2.08-1.99 (m, 1H). ¹³C NMR (126 MHz, DMSO) δ 172.8, 169.9, 166.9, 166.7, 165.5, 157.0, 154.9, 142.1, 137.1, 137.0, 133.0, 129.6, 129.3, 127.6, 123.1, 121.0, 120.3, 116.7, 116.1, 108.9, 69.6, 69.3, 68.8, 67.5, 64.4, 54.4, 52.2, 49.3, 48.8, 38.3, 30.9, 24.3, 22.0 (one resonance obscured by solvent, two resonances overlapping). HRMS (ESI-TOF) calculated for C₃₅H₃₉ClN₇O₉⁺ [M+H]⁺: 736.2492, observed 736.2447.

General procedure for AlphaScreen assay. His₉-tagged BPTF bromodomain was expressed and purified as described previously. His-tagged BRD9 and CECR2 were purchased from Reaction Biology (Cat. #s RD-11-214 and RD-11-210 for BRD9 and CECR2 respectively). The AlphaScreen assay procedures were adapted from the manufacturers protocol (PerkinElmer, USA). Nickel chelate (Ni-NTA) acceptor beads and streptavidin donor beads were purchased from PerkinElmer (Cat. #: 6760619M). The biotinylated Histone H4 KAc5,8,12,16 peptide was purchased from EpiCypher (Cat. #12-0034), with the sequence:

Ac-SGRGK(Ac)GGK(Ac)GLGK(Ac)GGAK(Ac)RHRKVLR-Peg(Biot).

All reagents were diluted in the assay buffer (50 mM HEPES-Na⁺ (ChemImpex), 100 mM NaCl (SigmaAldrich), 0.05% CHAPS (RPI), 0.1% BSA (SigmaAldrich), pH 7.4). The final assay concentrations of protein and biotinylated peptide used are shown in Table 2.

TABLE 2
Concentrations of protein and peptide used in AlphaScreen assays

| Protein | [Protein] nM | [Peptide] nM |
|---|---|---|
| BPTF | 30 | 100 |
| BRD9 | 60 | 100 |
| CECR2 | 60 | 100 |

3-fold serial dilutions were prepared with varying concentrations of the compounds and a fixed protein concentration, keeping the final DMSO concentration at 0.25%. 5 μL of these solutions were added to a 384-well plate (ProxiPlate-384, PerkinElmer). 5 μL of the biotinylated peptide solution was then added to the wells. 10 μL of pre-mixed nickel chelate acceptor beads and streptavidin donor beads were added to each well under low light conditions (<100 lux), to a final concentration of 20 μg/mL. The plate was sealed and incubated for 30 minutes in the dark. It was then read in AlphaScreen mode (excitation time=100 ms, integration time=300 ms, output: counts/s) using a Tecan Spark plate reader. Each compound was run in two technical replicates and the data was normalized against 0 μM inhibitor signal to obtain the % normalized AlphaScreen signal. IC₅₀ values were calculated in GraphPad Prism 9 using a sigmoidal 4-parameter logistic (4PL) curve fit.

General procedure for in vitro ternary complex assay. His-tagged CRBN-DDB1 protein was prepared following the procedure reported by Matyskiela et al. GST-BPTF was purchased from BPS Biosciences (cat #31134), while GST-BRD9, GST-CECR2, and GST-PCAF were purchased from Reactive Biology Corp (cat #RD-11-187, cat #RD-11-194, and cat #RD-11-259 respectively). All reagents were diluted in assay buffer comprising 25 mM HEPES, pH 7.4, 100 mM NaCl, 0.1% BSA, and 0.05% tween20. An ECHO 650 (Labcyte Inc.) acoustic dispenser was used to generate a 10-point dilution curve from DMSO stocks of the degraders directly into a 384-well OptiPlate (PerkinElmer, Cat. #6007290) giving a final DMSO concentration of 0.3%. Final concentrations of His-tagged CRBN-DDB1 and GST-tagged bromodomains used in the assay are shown in Table 3. AlphaScreen glutathione coated donor and nickel chelate acceptor beads were purchased from PerkinElmer (Cat. #6765300 and 6760141 respectively).

TABLE 3
Concentration of proteins used in AlphaScreen ternary complex assays

| | GST-tagged | |
|---|---|---|
| | Bromodomains | |
| Protein | (nM) | His-CRBN-DDB1 (nM) |
| PTF | 0 | 120 |
| RD9 | 40 | 80 |
| ECR2 | 40 | 80 |
| CAF | 40 | 80 |

Briefly, to a 384-well OptiPlate containing 5× degrader in triplicate was added 5 μL of a 5× solution of His-CRBN-DDB1 and GST-tagged bromodomain and then incubated at rt for 1 h. After incubation, 10 μL nickel chelate acceptor (20 μL/mL final concentration) and 10 μL glutathione donor beads (20 μL/mL final concentration) were added. The plate was sealed and mixed on a MixMate (eppendorf) for 1 h at rt and then luminescence detection was collected on an Envision plate reader (PerkinElmer).

In-cell NanoBRET assay. NanoBRET experiments were carried out using the NanoBRET CRBN Ternary Complex Starter Kit (Cat #ND2720) according to the manufacturer's protocol (Promega NanoBRET™ CRBN Ternary Complex Assay TM615).³⁸ All assays were run in 384-well format. HEK293T cells were transfected to a 1:100 donor:acceptor (Nluc:Halotag vectors) ratio for both BPTF-BD and BRD9-FL vectors (see supporting information for the design of plasmids). Cells were re-plated, treated with the HaloTag 618 Ligand and treated with compounds as described in the manufacturer's procedure. Data was collected on a Tecan Spark plate reader using the settings in Table 4.

TABLE 4
Tecan Spark settings for NanoBRET experiments

| | |
|---|---|
| 1) Shaking | Duration [s] 30 |
| (Linear) | Amplitude [mm] 1 |
| | Frequency [rpm] 1440 |
| 2) Mode: Luminescence Multi Color | |
| Donor Emission | Wavelength start = 445 nm |
| | Wavelength end = 470 nm |
| | Integration time = 300 ms |
| Acceptor Emission | Wavelength start = 610 nm |
| | Wavelength end = 700 nm |
| | Integration time = 300 ms |

Competition with monovalent inhibitor for ternary complex formation. For competition experiments, 10× solutions of compound 19 and MG-132 were prepared in Opti-MEM® I Reduced Serum Medium and pre-mixed in tubes. 10 μL of this solution was then added to each well. The plates were incubated for 30 minutes, followed by the addition of degrader compounds. The rest of the assay was carried out according to the technical manual referenced above.

Cell culture. HEK293T cells were grown in a humidified 5% CO₂ environment at 37° C. Cells were cultured in DMEM media (high-glucose, Gibco Cat. #11965-092) supplemented with 10% fetal-bovine serum (Cellgro Cat. #QB-110-001-101), Penicillin-Streptomycin (50 U/mL penicillin, 50 μg/mL streptomycin, Cellgro Cat. #15140-122). The cells were passaged to a 1:20 dilution by decanting suspended cells and dissociating adherent cells from cell culture flasks in 0.25% trypsin/EDTA (Gibco, Cat. #25200056) after 1 min incubation.

Western blotting. HEK293T cells were seeded at a density of 5×10⁵ cells per well in 6-well plates in 2.5 mL of medium. At 80-90% confluency, 250 μL of the medium was removed from each well and the cells were treated with compounds at the desired concentrations for indicated times, to a final DMSO concentration of 0.1% v/v. For collecting lysates, the medium was removed and the wells were rinsed with 1 mL of ice-cold PBS, followed by the addition of 100 μL cold RIPA buffer (ThermoFisher Cat. #89900) supplemented with cOmplete Mini Protease Inhibitor cocktail (Roche Cat. #11836153001). The plates were incubated on ice for 15 min. After high-speed centrifugation (15,000 g for 15 minutes), the supernatant was collected and protein concentrations were determined by the BCA assay (ThermoFisher Cat. #23227). The samples were normalized by total protein content, mixed with 4× NuPAGE LDS loading buffer (Invitrogen) and heated at 100° C. for 10 minutes.

Proteins were resolved by SDS-PAGE on NuPage 4-12% Bis-Tris (for CECR2, BRD9 and PCAF) or 3-8% Tris-acetate gels (for BPTF) (Invitrogen Cat. #NP0323BOX, EA03785BOX) and transferred to PVDF membrane (Bio-rad Cat. #1620174) using wet transfer for 60 minutes. Membranes were dried, blocked in TBS-T (Tris-buffered saline-T) containing 5% w/v nonfat dry milk for 1 h at room temperature. They were subsequently incubated with primary antibodies at dilutions and times in Table 5. After the membranes were washed five times with TBS-T, they were incubated with secondary antibodies at dilutions and times listed below. Finally, the membranes were washed five times in TBS-T and treated with SuperSignal West Dura substrates (ThermoFisher Cat. #34095) and imaged using a Bio-rad Chemi-Doc imaging system.

TABLE 5
Antibodies used for western blotting

| | | | | | | |
|---|---|---|---|---|---|---|
| | | Manu- | | | Incubation | Con- |
| Target | Species | facturer | Cat. # | Dilution | time | jugate |

Primary Antibodies

| | | | | | |
|---|---|---|---|---|---|
| BPTF | Rabbit | Millipore | MLL- | 1:1000 | 1 h RT or |
| | | Sigma | ABE24 | | overnight |
| | | | | | at 4° C. |
| CECR2 | Rabbit | LSBio | LS- | 1:500 | 1 h RT or |
| | | | C496852 | | overnight |
| | | | | | at 4° C. |
| BRD9 | Rabbit | Bethyl | A303- | 1:1000 | 1 h RT or |
| | | | 781A | | overnight |
| | | | | | 4° C. |
| PCAF | Rabbit | Cell | C14G9 | 1:1000 | 1 h RT or |
| | | Signaling | | | overnight |
| | | | | | 4° C. |
| Vinculin | Mouse | Invitrogen | 14-9777- | 1:2000 | 30 min RT |
| | | | 82 | | |
| β-actin | Mouse | Invitrogen | MA5- | 1:2000 | 30 min RT |
| | | | 11869 | | |

Secondary Antibodies

| | | | | | | |
|---|---|---|---|---|---|---|
| Mouse | Goat | Invitrogen | G-21040 | 1:10,000 | 1 h RT | HRP |
| Rabbit | Goat | Invitrogen | G-31460 | 1:1000 | 1 h RT | HRP |

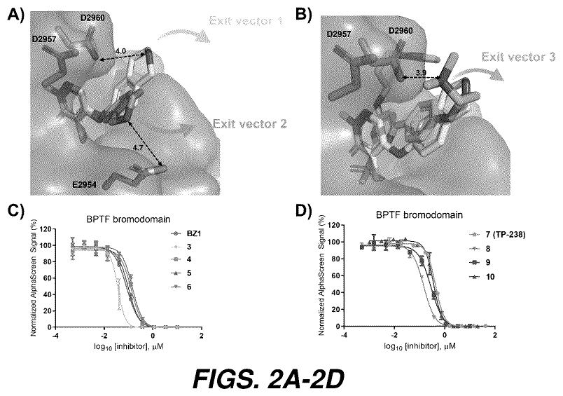

It has been previously reported that compounds 1 and 2 have high affinity pyridazinone-based ligands for the BPTF bromodomain. Through x-ray cocrystal structures, it was shown that with different positions of the amine in the tetrahydroisoquinoline ring, D2957, D2960 and E2954 in BPTF (PDB: 7RWQ and 7RWO) can be engaged. Therefore, for our first-generation pyridazinone degraders, a choice was made to attach linkers from the tetrahydroisoquinoline analogues, allowing us to explore two distinct exit vectors (FIG. 2A). Alkyl and PEG based linkers were chosen to assess any potential differences in ternary complex formation and cell permeability. Degraders 3-6 were synthesized according to Scheme 2, using pomalidomide-based cereblon-targeting E3 ligase ligands.

A crystal structure of a second inhibitor, TP-238, (compound 7) with BPTF (PDB ID: 7KDZ) has been disclosed. Overlaying the cocrystal structures of TP-238 and compound 1 (FIG. 2B) indicated that the pendant N(CH₃)₂ group in TP-238 provides a similar exit vector to the amine in compound 1, although the longer and flexible alkyl chain in TP-238 may provide more conformations in solution. Using TP-238, degraders 8-10 were synthesized. Compounds 8 and 9 are comprised of more rigid and polar piperazine linkers, with alkyl and PEG versions respectively. To study the effect of an alkyl linker in lieu of the piperazine ring, compound 10 was designed as the closest analogue of the parent TP-238 molecule.

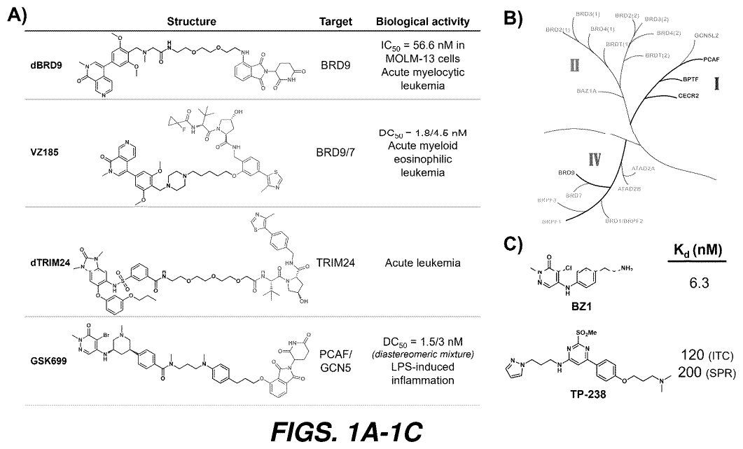

Biophysical characterization of pyridazinone and TP-238 based degraders. To validate our chosen exit vectors, an AlphaScreen assay was used to measure binding affinity of our degraders with the BPTF bromodomain. Heterobifunctional molecules from both scaffolds were high affinity binders of the BPTF (FIG. 1C and Table 6), establishing that our exit vectors do not significantly perturb binding to the BPTF bromodomain.

TABLE 6
AlphaScreen IC₅₀ values for inhibitors and degraders with BPTF, BRD9 and CECR2 bromodomains.

| | 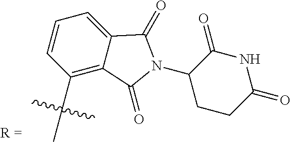 [SMILES: *c1cccc2c1C(=O)N(C1CCC(=O)NC1=O)C2=O] | BPTF BD IC₅₀ (nM)ª | BRD9 BD IC₅₀ (nM)^{b} | CECR2 BD IC₅₀ (nM)^{b} |
|---|---|---|---|---|
| 1 | 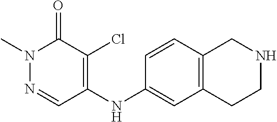 [SMILES: Cn1ncc(Nc2ccc3c(c2)CCNC3)c(Cl)c1=O] | 250 | 556 | 1670 |
| 2 | 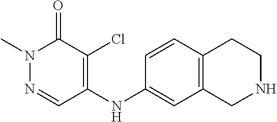 [SMILES: Cn1ncc(Nc2ccc3c(c2)CNCC3)c(Cl)c1=O] | 370 | 300 | 1300 |
| 3 | 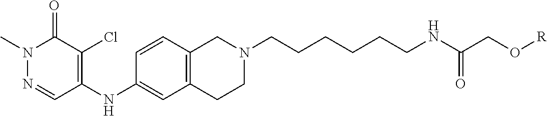 | 24 ± 9 | 21 | 53 |
| 4 | 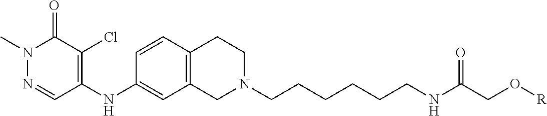 | 158 ± 55 | 98 | 485 |
| 5 | 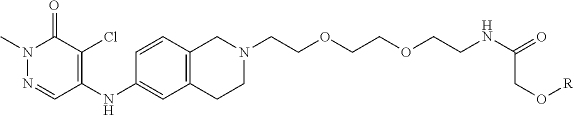 | 70 ± 23 | 63 | 225 |
| 6 | 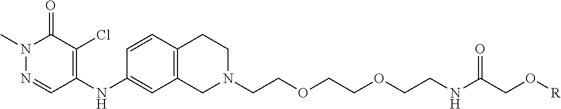 | 121 ± 38 | 18 | 1460 |
| 7 | 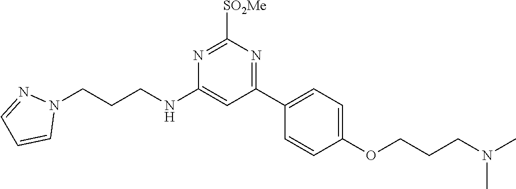 [SMILES: CN(C)CCCOc1ccc(-c2cc(NCCCn3cccn3)nc(S(C)(=O)=O)n2)cc1] | 430 | | 33 |
| 8 | 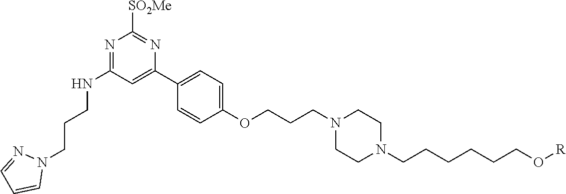 | 146 ± 12 | 844 (N = 1) | 24 (N = 1) |
| 9 | 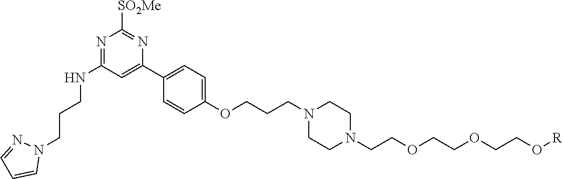 | 267 ± 8 | 996 (N = 1) | 33 (N = 1) |
| 10 | 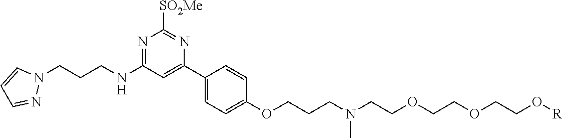 | 360 (N = 1) | ND | ND |

ªAverage of two technical replicates with N = 3. ^{b}Average of two technical replicates with N = 2 unless otherwise indicated. ND = not determined. Binding isotherms are shown in FIG. 2C.

The affinity of these compounds against the BRD9 and CECR2 bromodomains was further characterized, which are significant off-targets of the two scaffolds (Table 1). As observed previously with pyridazinone-based compounds, they retained binding to BRD9 and CECR2, although compound 4 and 6 demonstrated some attenuation in affinity to CECR2. Not surprisingly, the TP-238-derived molecules displayed the highest affinity to CECR2 and slightly weaker (x-x fold), binding to BPTF, consistent with the binding profile previously reported for the bromodomain-targeting ligand. In contrast, these compounds were low affinity binders for BRD9. The pyridazinone and pyrimidine-based scaffolds therefore provided us with two unique selectivity profiles to target representative members of the class I and class IV bromodomain families.

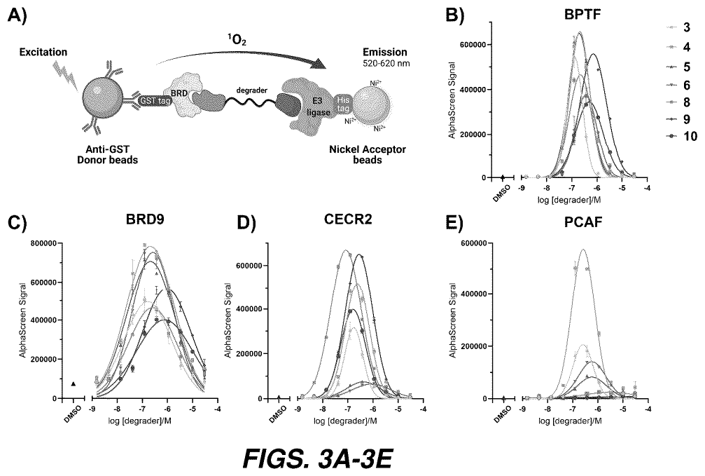

In vitro ternary complex formation via AlphaScreen. The stability of the ternary complex is critical for degradation by heterobifunctional molecules. A ternary complex formation in vitro using an AlphaScreen-based assay (FIG. 3A) was therefore studied. In this experiment, anti-GST donor beads and nickel acceptor beads were paired with GST-tagged bromodomains (BPTF, BRD9, CECR2 and PCAF) and His-tagged CRBN. The AlphaScreen signal was measured at varying degrader concentrations and the amplitude of the maximum point on the curve was used as an indicator of ternary complex stability.

In this assay format, all pyridazinone-based degraders showed high ternary complex formation with BPTF (FIG. 3B), consistent with the high affinity of the pyridazinone for this target. To our surprise, while compound 4 seemed to be promiscuous, compound 3 showed a drop in signal for BRD9, CECR2 and PCAF (FIGS. 3C-E), indicating that the exit vector 1 (FIG. 2A) in the alkyl linker series may help bias selectivity for BPTF over the other bromodomains tested in this assay. A similar albeit minor effect was observed in the PEG linker series in the case of PCAF (FIG. 3E) but not BRD9 or CECR2. Comparing the alkyl and PEG linkers, compounds 5 and 6 with PEG linkers generally seemed to form less stable ternary complexes for CECR2 and PCAF, compared to BPTF and BRD9. Since these compounds do not demonstrate any significant loss of affinity with the bromodomain target (Table 1), it was hypothesizes that there may be additional positive or negative cooperativity effects during ternary complex formation contributing to this selectivity.

In contrast, a significant difference between the alkyl and PEG linkers was not observed for the TP-238 analogues. Both compounds 8 and 9 showed similar ternary complex behavior with BPTF, BRD9 and CECR2. The signals for compound 10 were generally lower across all the proteins tested, highlighting the importance of the more rigid piperazine linker framework as opposed to alkyl chains in this scaffold. There was therefore a focus on compounds 8 and 9 for further studies.

Comparing our pyridazinone and pyrimidine scaffolds, the pyridazinone degraders typically showed more potent ternary complex formation for BPTF and BRD9, while the TP-238 degraders were more potent for CECR2. This was consistent with the selectivity profile of the bromodomain-targeting moiety of these degraders. Encouragingly, apart from compound 4, all of the other degraders demonstrated a significant loss of ternary complex stability for PCAF (and most likely GCN5 due to their high sequence similarity), indicating selectivity over this important class I bromodomain target.

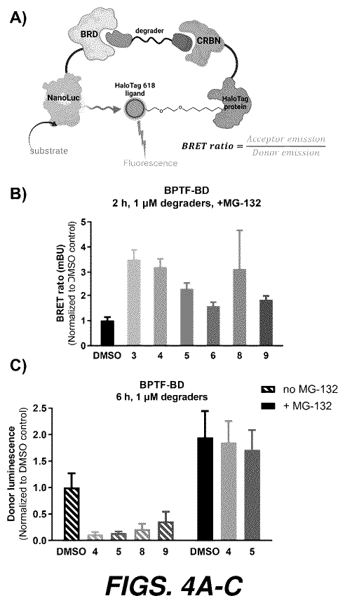

In-cell ternary complex formation and degradation of BPTF-BD via NanoBRET. After establishing in vitro ternary complex formation, a NanoBRET ternary complex assay was optimized to rank-order degraders in cells (FIG. 4A). This assay also serves as an indirect indicator of cellular permeability, which is often a major challenge for heterobifunctional degraders. Both N- and C-terminal fusions of Nanoluciferase (Nluc) and BPTF-BD with 11-amino acid linkers were designed. The vectors were transfected into HEK293T cells and compounds 4 and 5 were dosed at 1 μM for 2 h in cells pre-treated with the proteosome inhibitor MG-132. It was found that the C-terminal fusion construct showed ternary complex formation while the N-terminal fusion did not with either compound. The C-terminal Nluc-BPTF-BD fusion construct was therefore used for subsequent experiments. Degraders were tested for ternary complex formation at 1 μM (FIG. 4B). In this assay, a trend was observed within the pyridazinone degrader series, where the alkyl linker compounds (3 and 4) demonstrate a higher BRET ratio (3.5- and 3.2-fold increase over DMSO respectively) compared to the PEG linkers (2.3- and 1.6-fold for compounds 5 and 6). For TP-238 series, a higher BRET signal was observed for compound 8 (3.1-fold over DMSO control) compared to compound 9 (1.8-fold), providing more evidence that the alkyl linkers appear to be favored. These data show that our compounds are highly cell-permeable in this assay, making them useful starting points for further SAR.

The degradation of Nluc-BPTF construct was then monitored by measuring the total donor luminescence after treatment of transfected HEK293T cells with 1 μM of the degraders for 6 h. A significant decrease was observed in donor luminescence with both our pyridazinone and TP-238 degraders compared to the untreated DMSO control, indicating degradation of the construct (FIG. 4C). To validate the proteosomal-dependence of this degradation activity, cells were pre-treated with 10 μM MG-132, a proteosome inhibitor, which resulted in rescue of degradation, with some additional stabilization of the donor signal.

Since luciferase enzyme-based assays are prone to interference due to potential inhibitors, any off-target effects of molecules described herein on the Nluc-BPTF construct was a potential concern. To demonstrate that these degraders bind to the BPTF bromodomain and not Nluc, treated transfected HEK293T cells were pre-with the monovalent BPTF inhibitor 19 that were previously characterized. This was tested in the ternary complex assay format and observed a dose-dependent dissociation of the ternary complex with increasing concentrations of 19. This further validated that the ternary complex formation is BPTF bromodomain-dependent.

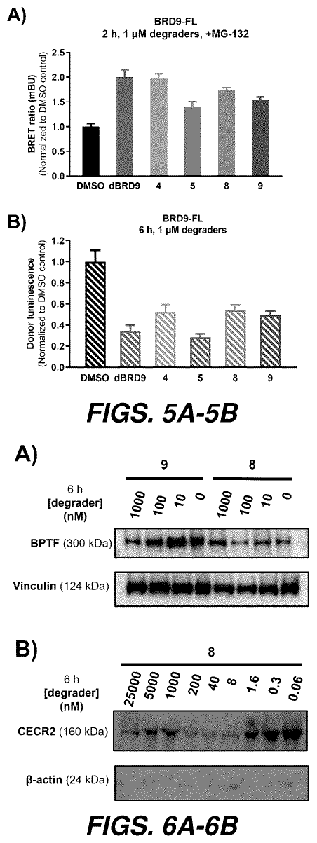

In-cell ternary complex formation and degradation of full-length BRD9 via NanoBRET. The compounds described herein were characterized for ternary complex formation and degradation of full-length BRD9 using NanoBRET. BRD9 is a reported off-target for the pyridazinone scaffold and TP-238. Due to its smaller size (75 kDa) compared to full-length BPTF (˜300 kDa), full-length (FL) BRD9 was expected to be more amenable to transfection and easier to study in this assay compared to full-length BPTF. dBRD9 could also be used as a positive control to validate our assay in this system. For the pyridazinone degraders, a similar trend was observed to BPTF ternary complex data, where compound 4 showed a higher BRET signal than 5, which was comparable in magnitude to dBRD9 (2-fold over DMSO control) (FIG. 5A). For the TP-238 series, the BRET ratios were slightly lower than dBRD9 (˜1.5-fold over DMSO), which is consistent with the lower affinity of the TP-238 ligand for BRD9, although given the data in Table 1, it was expected to show a much lower signal. It is unclear if the full-length BRD9 shows some additional affinity for this scaffold in the context of this experiment. Degradation of the Nluc-BRD9-FL construct was monitored under similar conditions as our BPTF study. Encouragingly, both compound 4 and 5 caused degradation of full-length BRD9, comparable to the dBRD9 control (FIG. 5B). Consistent with their ternary complex behavior, compounds 8 and 9 also degrade BRD9-FL, albeit not as potently as dBRD9. These studies confirmed that both series of degrader molecules can engage full-length bromodomain-containing proteins in cells and induce their degradation. They also established the robustness of our NanoBRET assay, indicating that it is applicable to multiple bromodomain-containing constructs.

Degradation by Western Blotting. With well-characterized compounds in hand, their ability to degrade endogenous BPTF in HEK293T cells was tested. The 6 h treatment condition from a NanoBRET assay was used and monitored the total amount of BPTF in lysates through western blotting. Surprisingly, our pyridazinone degraders did not show any degradation of endogenous BPTF or a second Class I bromodomain-containing protein, PCAF under these conditions. In contrast, the TP-238 series demonstrated a moderate effect between x-x μM concentrations against BPTF (FIG. 6A). Taken together with our NanoBRET data, it was hypothesized that the minimal effect of these compounds on full-length BPTF in endogenous systems may be a cell-line dependent effect and the levels of functional BPTF available for degradation. In cells, endogenous BPTF is a subunit of the NURF complex, which may hinder its accessibility by heterobifunctional degraders. The moderate effect observed via the TP-238 scaffold hints towards longer, pre-organized linkers (such as the piperazine moiety in compounds 8 and 9) which may be able to improve the activity of BPTF degraders.

Due to our challenges with observing degradation of BPTF through western blotting, the main off-target proteins of the two scaffolds were studied. TP-238 is a high affinity inhibitor of CECR2 so compounds 8 and 9 were expected to be potent degraders of CECR2. Encouragingly, significant degradation of CECR2 between 8-200 nM concentrations (FIG. 6B) in HEK293T cells after 6 h treatment was observed with compounds 8 and 9.

### BRD9 Blots.

The design of heterobifunctional degraders targeting class I and BRD9 bromodomains, using two different scaffolds with distinct selectivity profiles are described. Exit vectors based on tetrahydroisoquinoline-substituted pyridazinones were explored, evaluating both alkyl and PEG linkers, and a rigid piperazine linker tethered to the TP-238 scaffold. Using an in vitro AlphaScreen assay to assess ternary complex formation, it was discovered that several of the pyridazinone analogues demonstrate a selectivity bias for BPTF and BRD9 over PCAF and CECR2. For the TP-238 degraders, the piperazine-based linker design is found to be more potent in this ternary complex analysis. An in-cell NanoBRET assay was used to show that the degraders (e.g., the compounds of Formula (I)) are cell-permeable and form ternary complexes with nanoluciferase-fused BPTF bromodomain and full-length BRD9 constructs. In the absence of a proteosome inhibitor, these fusion constructs are also degraded when treated with our degrader scaffolds. However, it was found that pyridazinone degraders do not show any degradation activity for endogenous BPTF through western blotting. In contrast, the TP-238 degraders demonstrate a moderate effect on BPTF levels and a strong dose-dependent attenuation of CECR2 levels via western blotting. These results indicate different levels of degradability between class I and IV bromodomain-containing proteins. Further optimization of our first-generation degraders and a more extensive study of various model cell lines will be evaluated for targeting these important nucleosome remodeling complexes.

The design of heterobifunctional degraders targeting class I and BRD9 bromodomains, using two dif-ferent scaffolds with distinct selectivity profiles, are described. Exit vectors based on tetrahydroisoquinoline-substituted pyridazinones, evaluating both alkyl and PEG linkers, and a rigid piperazine linker tethered to the TP-238 scaffold were explored. Using an in vitro AlphaScreen assay to assess ternary com-plex formation, it was discovered that several of the pyridazinone analogues demonstrate a selectivity bias for BPTF and BRD9 over PCAF and CECR2. For the TP-238 de-graders, the piperazine-based linker design is found to be more potent in this ternary complex analysis. An in-cell NanoBRET assay was used to show that degraders (e.g., compounds of the Formula (I)) are cell-permeable and form ternary complexes with nanoluciferase-fused BPTF bromodomain and full-length BRD9 constructs. In the absence of a proteosome inhibitor, these fusion constructs are also degraded when treated with our degrader scaffolds. However, it was found that pyridazinone degraders do not show any degradation activity for endogenous BPTF through western blotting. In contrast, the TP-238 degraders demonstrate a moderate effect on BPTF levels and a strong dose-dependent attenuation of CECR2 levels via western blotting. These results indicate different levels of degradability between class I and IV bromodomain-containing proteins. Further optimization of our first-generation degraders and a more extensive study of various model cell lines will be evaluated for targeting these important nucleosome remodeling complexes.

1. A compound of the Formula (I):
A-L¹-B Formula (I)
or a pharmaceutically acceptable salt, polymorph, prodrug, solvate or clathrate thereof, wherein A is a ligand for the bromodomains of at least one of BPTF, CECR2, BRD9, and PCAF/GCN5; B is an E3 ligase ligand; and L¹ is a linker.

2. The compound of claim 1, wherein A is a group of the formula X—N(H)—Z, wherein L¹ is covalently attached to X, wherein:
Z is a group of the formula: 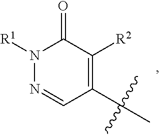
wherein R¹ is H or alkyl and R² is halo; and
X is a heterocyclyl group.

3. The compound of claim 1, wherein X is a group of the formula: 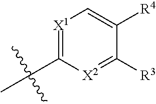
wherein X¹ and X² are each independently CH or N; and R² and R³, together with the carbon atoms to which they are attached, form a heterocyclyl group; or
X is a group of the formula: 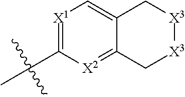
wherein X¹ and X² are each independently CH or N; and each X³ is, independently, CH₂ or N provided that one of X³ is N and X³ is attached to L¹.

4. (canceled)

5. The compound of claim 3, wherein X is a group of the formula: 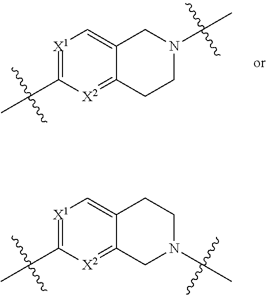
wherein X¹ and X² are each independently CH or N.

6. (canceled)

7. The compound of claim 2, wherein X—Y—Z is a group of the formula:

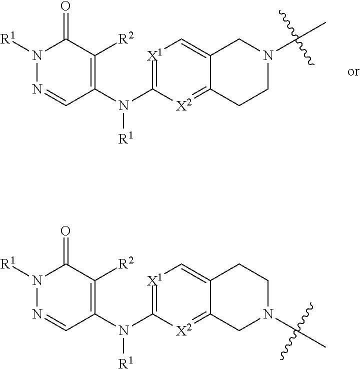

8. The compound of claim 1, wherein A is a group of the formula X¹—Z²—Y¹—Z¹, wherein L¹ is covalently attached to X¹, wherein:
Z¹ is a five-membered heterocyclyl group;
Y¹ is alkyl-NR¹—, wherein R¹ is H or alkyl;
Y² is a six-membered heterocyclyl group; and
X¹ is aryl-O—.

9. The compound of claim 8, wherein Z¹ is a group of the formula:

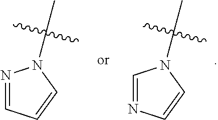 [SMILES: *n1cccn1 ; *n1ccnc1]

10. The compound of claim 8, wherein Z² is a group of the formula: 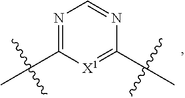
wherein X¹ is CH or N.

11. The compound of claim 8, wherein X¹ is:

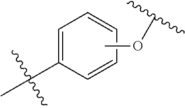

12. The compound of claim 1, wherein L¹ is acyl, amido, alkyl, alkenyl, alkynyl, heterocyclyl, and combinations thereof, optionally interrupted by one or more heteroatoms.

13. The compound of claim 12, wherein the one or more heteroatoms are selected from the group consisting of —O—, —NR¹— (wherein R¹ is H or alkyl), and —S(O)ₙ— (wherein n is 0, 1 or 2).

14. The compound of claim 1, wherein L¹ comprises alkyl, -(alkyl-O)ₓ—, -(alkyl-NR¹)ₓ— (wherein x is an integer from 1 to 10), —NR¹—C(O)— (wherein R¹ is H or alkyl), a six-membered heterocyclyl group, and combinations thereof.

15. The compound of claim 1, wherein L¹ is: 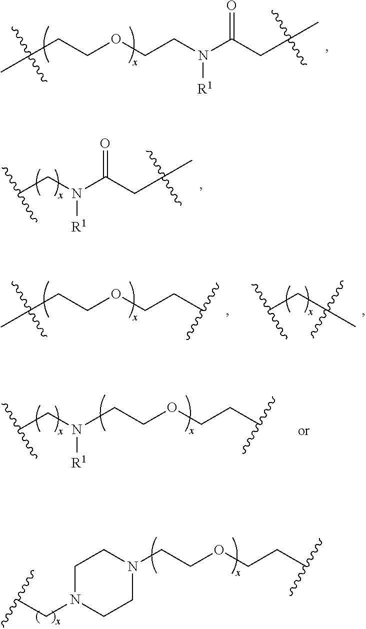
or combinations thereof, wherein R¹ is H or alkyl and each x is, independently, an integer from from 1 to 10.

16. The compound of claim 1, wherein B is a group of the formula:

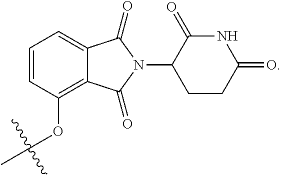 [SMILES: *Oc1cccc2c1C(=O)N(C1CCC(=O)NC1=O)C2=O]

17. The compound of claim 1, wherein the compound is a compound of the formula: 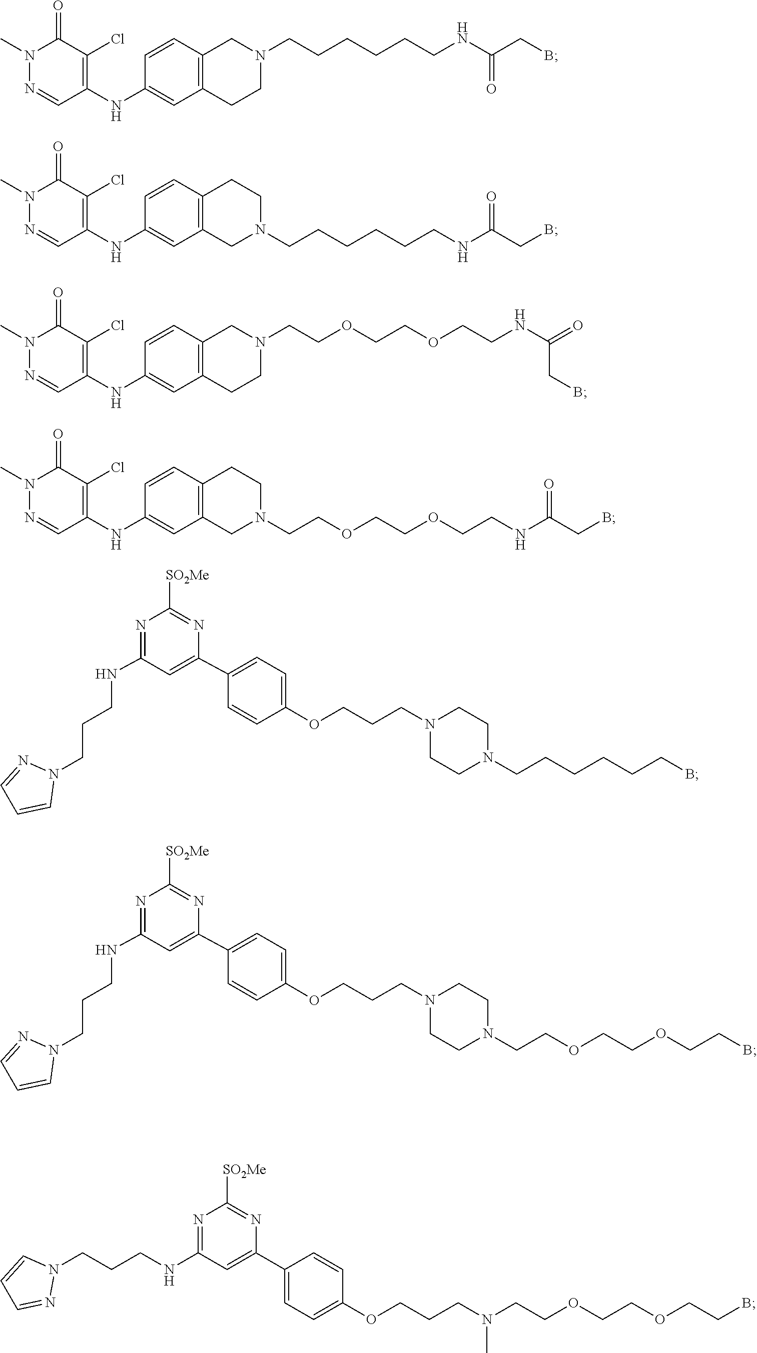
or a pharmaceutically acceptable salt, polymorph, prodrug, solvate or clathrate thereof.

18. (canceled)

19. A pharmaceutical composition comprising one or more compounds of any preceding claim and one or more pharmaceutically acceptable excipients.

20. A method of modulating the activity of at least one of BPTF, CECR2, BRD9, and PCAF/GCN5 comprising administering a therapeutically effective amount of a compound of any preceding claim, or a pharmaceutical composition comprising said compound, to a subject in need thereof.

21. A method for treating cancer comprising administering a therapeutically effective amount of one or more compounds of any preceding claim, or a pharmaceutical composition comprising said compound, to a subject in need thereof.

22. The method of claim 21, wherein the cancer is breast cancer, non-small-cell lung cancer, colorectal cancer or high-grade gliomas.

23. (canceled)

24. A method for treating inflammation comprising administering a therapeutically effective amount of one or more compounds of any preceding claim, or a pharmaceutical composition comprising said compound, to a subject in need thereof.
